(19) United States
(12) Patent Application Publication
(10) Pub. No.: US 2005/0085644 A1
(43) Pub. Date: Apr. 21, 2005

(54) Novel 3-aryl-2,5-dihydroxy-1,4-benzoquinone derivatives, their preparation method and pharmaceutical compositions containing same

(76) Inventors: Alexandra Dassonville, Reims (FR); Anne Breteche, Orvault (FR); Muriel Duflos, La Croix Rouge (FR); Guillaume Le Baut, Saint Sebastien (FR); Bruno Pfeiffer, Saint Leu La Foret (FR); Pierre Renard, Le Chesnay (FR); Nigel Levens, Vaucresson (FR); Bernadette Husson-Robert, Saint Apollinaire (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/503,527

(22) PCT Filed: Feb. 4, 2003
(86) PCT No.: PCT/FR03/00331

(30) Foreign Application Priority Data
Feb. 6, 2002 (FR) 02/01409

(51) Int. Cl. C07C 50/38; C07D 213/46; C07D 207/00

(52) U.S. Cl. 546/340; 548/400; 552/298

(57) ABSTRACT

Compound of formula (I): 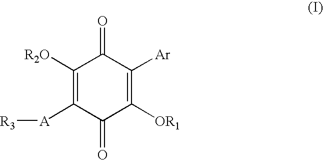 wherein:
R₁ and R₂, which may be identical or different, each represents hydrogen, acyl or alkyl, Ar represents aryl or heteroaryl,
   A represents a group selected from: 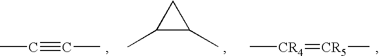
   R₄ represents hydrogen or alkyl,
   R₅ represents hydrogen, alkyl, aryl or heteroaryl,
R₃ represents aryl, heteroaryl, dicyclopropylmethyl or benzhydryl, or A-R₃ represents an optionally substituted naphthyl group, with the exclusion of 2,5-dihydroxy-3-(4-methoxyphenyl)-6-(2-phenylethenyl)-1,4-benzoquinone, 2,5-dihydroxy-3-(4-methoxyphenyl)-6-(2-naphthyl)-1,4-benzoquinone and 2,5-dihydroxy-3-(2-naphthyl)-6-phenyl-1,4-benzoquinone. Medicinal products containing the same which are useful in the treatment of diseases associated with a deregulation of glycaemia.

The present invention relates to new 3-aryl-2,5-dihydroxy-1,4-benzoquinone compounds, to a process for their preparation, to pharmaceutical compositions containing them and to the use thereof as anti-diabetics.

A 3-aryl-2,5-dihydroxy-6-(2-phenylethenyl)-1,4-benzoquinone has been described in the journal Liebigs Ann. Chem. 1986, 195-204 for its potential anti-tumour activity and its anti-oxidant activity.

The compounds of the present invention exhibit insulinomimetic properties, such as an increase in the autophosphorylation of the insulin receptor and of protein kinase B.

Insulin resistance is a very complex syndrome exhibiting deficiencies at different levels of the intracellular signaling cascade of insulin. In addition to a decrease in the number of insulin receptors (Kahn et al., Mechanism of action of hormones that act at the cell surface, 8^{th} edition, WB 91-134, Saunders, Philadelphia, 1992), there is clearly an alteration in the kinase activity of the insulin receptor. That post-receptor deficiency occurs on the one hand in the phosphorylation of the tyrosine of IRS1 and on the other hand in the IRS1/PI3 kinase interaction (Y. Le Marchand-Brustel, Exp. Clin. Endocrinol. Diabetes, 1999, 107, 126-132), thus limiting the activation of protein kinase B, key enzyme in the utilisation of glucose (Burgering et al., Nature, 1995, 376 (6541), 599-602) and in apoptosis (Franke T. F., Cell, 1997, 88, 435-437).

The properties of the compounds of the present invention in relation to the insulin receptor and protein kinase B thus makes them of great interest for the treatment of diseases associated with a deregulation of glycaemia.

It will be possible for them to be used especially in the treatment of diabetes (type I or type II diabetes).

More specifically, the present invention relates to compounds of formula (I): 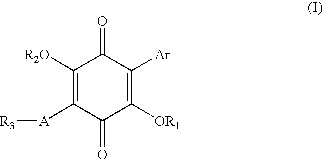
wherein:
R₁ and R₂, which may be identical or different, each represents a hydrogen atom or a linear or branched (C₁-C₆)acyl or linear or branched (C₁-C₆)alkyl group,
Ar represents an aryl or heteroaryl group,
A represents a group selected from: 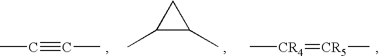
   R₄ substitutes the carbon atom bonded to the benzoquinone ring and represents a hydrogen atom or a linear or branched (C₁-C₆)alkyl group,
   R₅ substitutes the carbon atom bonded to the radical R₃ and represents a hydrogen atom or a group selected from linear or branched (C₁-C₆)alkyl, aryl and heteroaryl,
R₃ represents an aryl, heteroaryl, dicyclopropylmethyl or benzhydryl group, or A-R₃ represents an optionally substituted naphthyl group, and in that case Ar represents an aryl group,
   and to their stereoisomers, where they exist, and also to addition salts thereof with a pharmaceutically acceptable acid or base,
   with the exclusion of 2,5-dihydroxy-3-(4-methoxyphenyl)-6-(2-phenylethenyl)-1,4-benzoquinone, 2,5-dihydroxy-3-(4-methoxyphenyl)-6-(2-naphthyl)-1,4-benzoquinone and 2,5-dihydroxy-3-(2-naphthyl)-6-phenyl-1,4-benzoquinone.

An aryl group is to be understood as phenyl, biphenylyl, naphthyl or tetrahydronaphthyl, each of those groups optionally being substituted by one or more identical or different atoms or groups selected from the halogen atoms and the groups linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl, (C₁-C₂)alkylenedioxy and phenyloxy.

An optionally substituted naphthyl group is to be understood as a naphthyl group that is unsubstituted or substituted by one or more identical or different atoms or groups selected from the halogen atoms and the groups linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl, (C₁-C₂)alkylenedioxy and phenyloxy.

A heteroaryl group is to be understood as an aromatic mono- or bi-cyclic group having from 5 to 12 ring members and containing one, two or three hetero atoms selected from oxygen, nitrogen and sulphur, wherein the heteroaryl may optionally be substituted by one or more identical or different atoms or groups selected from the halogen atoms and the groups linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl, (C₁-C₂)alkylenedioxy and phenyloxy. Among the heteroaryl groups there may be mentioned, without implying any limitation, the groups thienyl, pyridyl, furyl, pyrrolyl, imidazolyl, oxazolyl, isoxazolyl, thiazolyl, isothiazolyl, quinolyl, isoquinolyl, pyrimidinyl.

A stereoisomer is to be understood as a double-bond geometric isomer or an optical isomer.

An advantageous variant according to the invention concerns compounds of formula (I): 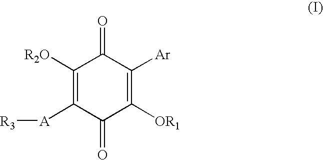
wherein:
R₁ and R₂, which may be identical or different, each represents a hydrogen atom or a linear or branched (C₁-C₆)acyl or linear or branched (C₁-C₆)alkyl group,
R₃ represents an aryl, heteroaryl, dicyclopropylmethyl or benzhydryl group,
Ar represents an aryl or heteroaryl group,
A represents a group selected from: 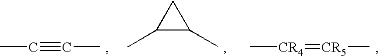
   R₄ substitutes the carbon atom bonded to the benzoquinone ring and represents a hydrogen atom or a linear or branched (C₁-C₆)alkyl group,
   R₅ substitutes the carbon atom bonded to the radical R₃ and represents a hydrogen atom or a group selected from linear or branched (C₁-C₆)alkyl, aryl and heteroaryl,
      and to their stereoisomers, where they exist, and also to addition salts thereof with a pharmaceutically acceptable acid or base,
      with the exclusion of 2,5-dihydroxy-3-(4-methoxyphenyl)-6-(2-phenylethenyl)-1,4-benzoquinone.

Another variant according to the invention concerns compounds of formula (I): 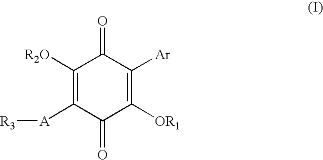
wherein:
R₁ and R₂, which may be identical or different, each represents a hydrogen atom or a linear or branched (C₁-C₆)acyl or linear or branched (C₁-C₆)alkyl group,
Ar represents an aryl group,
A-R₃ represents an optionally substituted naphthyl group,
   and to their stereoisomers, where they exist, and also to addition salts thereof with a pharmaceutically acceptable acid or base,
   with the exclusion of 2,5-dihydroxy-3-(4-methoxyphenyl)-6-(2-naphthyl)-1,4-benzoquinone and 2,5-dihydroxy-3-(2-naphthyl)-6-phenyl-1,4-benzoquinone.

Preferred R₁ and R₂ groups are the hydrogen atom.

Ar advantageously represents an aryl group and more especially an unsubstituted or substituted phenyl or naphthyl group.

Even more especially, Ar represents a phenyl or naphthyl group, each of those groups being unsubstituted or substituted by a halogen atom, such as chlorine or bromine for example.

Preferred A-R₃ groups are the unsubstituted or substituted naphthyl group and the arylethenyl group, and more especially the unsubstituted or substituted phenylethenyl group. Substituents of the naphthyl and phenylethenyl groups are preferably halogen atoms, such as chlorine, bromine or fluorine.

Even more especially, the invention relates to the following compounds of formula (I):
2-(4-chlorophenyl)-3,6-dihydroxy-5-(2-naphthyl)-1,4-benzoquinone,
2-(4-bromophenyl)-3,6-dihydroxy-5-(2-naphthyl)-1,4-benzoquinone,
2-[(E)-2-(4-fluorophenyl)ethenyl]-3,6-dihydroxy-5-phenyl-1,4-benzoquinone,
2-(4-chlorophenyl)-3,6-dihydroxy-5-[(E)-2-phenylethenyl]-1,4-benzoquinone,
2,5-dihydroxy-3-phenyl-6-[(E)-2-phenylethenyl]-1,4-benzoquinone,
2-(4-bromophenyl)-3,6-dihydroxy-5-[(E)-2-phenylethenyl]-1,4-benzoquinone,
2,5-dihydroxy-3-[(E)-1-methyl-2-phenylethenyl]-6-phenyl-1,4-benzoquinone,
and 2-(3-chlorophenyl)-3,6-dihydroxy-5-[(E)-2-phenylethenyl]-1,4-benzoquinone.

The stereoisomers and the addition salts with a pharmaceutically acceptable acid or base of the preferred compounds of the invention form an integral part of the invention.

Amongst the pharmaceutically acceptable bases there may be mentioned, without implying any limitation, sodium hydroxide, potassium hydroxide, triethylamine, tert-butylamine.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid.

The invention relates also to a new 3-aryl-2,5-dihydroxy-1,4-benzoquinone compound, which is 2-(4-bromophenyl)-3,6-dihydroxy-5-phenyl-1,4-benzoquinone, and to addition salts thereof with a pharmaceutically acceptable base.

The invention relates also to a process for the preparation of compounds of formula (I), which process is characeterised in that a compound of formula (II): 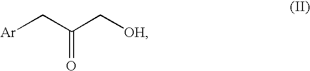
wherein Ar is as defined for formula (I), is reacted with a compound of formula (III): 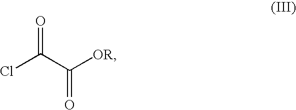
wherein R represents a linear or branched (C₁-C₆)alkyl group,
to yield a compound of formula (IV): 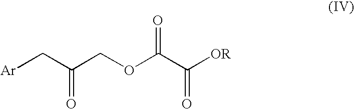
wherein Ar and R are as defined hereinbefore, which is cyclised under basic conditions then, if desired, with an acylation or alkylation reagent, to yield a compound of formula (V): 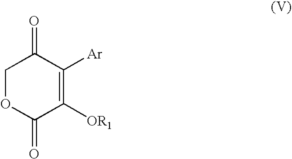
wherein Ar is as defined hereinbefore and R₁ is as defined for formula (I),
which is reacted with a compound of formula (VI): 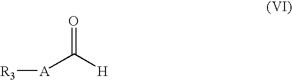
wherein A and R₃ are as defined for formula (I),
to yield a compound of formula (VII): 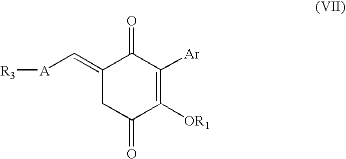
wherein A, Ar, R₁ and R₃ are as defined hereinbefore,
which is subjected to basic conditions and then, if desired, to the action of an acylation or alkylation reagent, to yield a compound of formula (I),
which is purified according to a conventional purification technique and is optionally separated into the stereoisomers according to a conventional separation technique.

The compounds of formula (II) can be obtained either by reaction of a compound of formula (VIII): 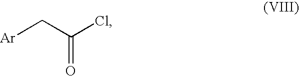
wherein Ar is as defined for formula (I),
with tris(trimethylsilyloxy)ethylene in the presence of a Lewis acid, or by reaction of a compound of formula (IX): 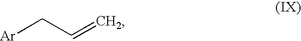
wherein Ar is as defined for formula (I), with peracetic acid in the presence of osmium trichloride.

In addition to being new, the compounds of the present invention exhibit valuable pharmacological properties. They have insulinomimetic properties which render them useful in the treatment of diseases associated with a deregulation of glycaemia, such as type I or type II diabetes.

The invention relates also to pharmaceutical compositions comprising as active ingredient at least one compound of formula (I) with one or more appropriate inert, non-toxic excipients. Amongst the pharmaceutical compositions according to the invention there may be mentioned more especially those which are suitable for oral, parenteral (intravenous, intramuscular or subcutaneous) or nasal administration, tablets or dragees, sublingual tablets, gelatin capsules, lozenges, suppositories, creams, ointments, dermal gels, injectable preparations, drinkable suspensions, etc.

The useful dosage is adaptable in accordance with the nature and severity of the disorder, the administration route and also the age and weight of the patient and any associated treatments. The dosage ranges from 0.5 mg to 2 g per 24 hours in one or more administrations.

The Examples which follow illustrate the invention but do not limit it in any way.

The starting materials used are known products or products prepared according to known preparation procedures.

The structures of the compounds described in the Examples were determined according to customary spectrometric techniques (infra-red, NMR, mass spectrometry).

### EXAMPLE 1

### 2,5-Dihydroxy-3-phenyl-6-[(E)-2-phenylethenyl]-1,4-benzoquinone

### Step A: 1-Hydroxy-3-phenylacetone

0.3 mmol of hydrated osmium trichloride and then slowly, dropwise, 20 mmol of a 30% peracetic acid solution in ethyl acetate, are added to 10 mmol of allylbenzene dissolved in a mixture of acetonitrile, dichloromethane and water. After the addition, stirring is carried out for 3 hours at ambient temperature and then the reaction mixture is poured into an aqueous 5% sodium bisulphite solution. After extraction with dichloromethane, the organic phases are combined, washed, dried and evaporated. The residue obtained is purified by chromatography on silica (eluant:hexane/ethyl acetate 7/3) to yield the expected product.

Melting point: 42-43° C.

### Step B: (2-Oxo-3-phenylpropyl) Ethyl Oxalate

11.6 mmol of triethylamine and then 10.4 mmol of ethyl oxalyl choride are added at 0° C. to 10 mmol of the compound obtained in the above Step in solution in tetrahydrofuran. After stirring for 3 hours, the mixture is extracted with ethyl acetate, and then the organic phase is washed, dried and evaporated to yield the expected product in the form of an oil.

### Step C: 3-Hydroxy-4-phenylpyran-2,5-dione

10 mmol of the compound obtained in the above Step in solution in dimethylformamide are slowly added dropwise, at −20° C., to 20.5 mmol of 1,8-diazabicyclo[5.4.0]undec-7-ene in solution in dimethylformamide. After stirring for 2½ hours at −15° C., the reaction mixture is slowly poured into a 1M hydrochloric acid solution at 0° C. The precipitate formed is filtered off, washed and then dried to yield the expected product.

Melting point: 175-176° C.

### Step D: 3-Hydroxy-6-phenylpropenylidene-4-phenylpyran-2,5-dione

10 mmol of cinnamaldehyde are added to 10 mmol of the compound obained in the above Step in glacial acetic acid. The reaction mixture is then heated at 60° C. until dissolved, and then a few drops of concentrated hydrochloric acid are added and the temperature is brought to 90° C. After stirring for 2 hours, the mixture is cooled to 0° C. and a 1/1 mixture of ether and hexane is added. The precipitate obtained is filtered off and then dried to yield the expected product.

Melting point: 231-232° C.

### Step E: 2,5-Dihydroxy-3-phenyl-6-[(E)-2-phenylethenyl]-1,4-benzoquinone

100 ml of a 30% by weight sodium methanolate solution in methanol are added at ambient temperature to 10 mmol of the compound obtained in the above Step in suspension in methanol. After stirring for 15 minutes, the reaction mixture is slowly poured into a 1M hydrochloric acid solution at 0° C. The precipitate is filtered off and then washed and dried to yield the expected product.

Melting point: 259-260° C.

Mass spectometry: MS m/z (%)=318.20 (100), 199.15 (40), 115.10 (36).

### EXAMPLE 2

### 2,5-Dihydroxy-3-phenyl-6-[(E)-2-(4-chlorophenyl)-ethenyl]-1,4-benzoquinone

The expected product is obtained in accordance with the procedure described in Example 1 with the replacement of cinnamaldehyde with 4-chloro-cinnamaldehyde in Step D.

Melting point: 244-245° C.

Mass spectometry: MS m/z (%)=325.7 (22), 351.7 (100), 235.86 (8), 323.87 (32).

### EXAMPLE 3

### 2,5-Dihydroxy-3-phenyl-6-[(E)-2-(4-bromophenyl)-ethenyl]-1,4-benzoquinone

The expected product is obtained in accordance with the procedure described in Example 1 with the replacement of cinnamaldehyde with 4-bromo-cinnamaldehyde in Step D.

Melting point: 240-241° C.

Mass spectometry: MS m/z (%)=397.35 (100), 396.95 (46)

### EXAMPLE 4

### 2,5-Dihydroxy-3-phenyl-6-(phenylethynyl)-1,4-benzoquinone

The expected product is obtained in accordance with the procedure described in Example 1 with the replacement of cinnamaldehyde with 3-phenyl-2-propynal in Step D.

### EXAMPLE 5

### 2-[(1E)-3,3-Dicyclopropyl-1-propenyl]-3,6-dihydroxy-5-phenyl-1,4-benzoquinone

The expected product is obtained in accordance with the procedure described in Example 1 with the replacement of cinnamaldehyde with (2E)-4,4-dicyclopropyl-2-butenal in Step D.

### EXAMPLE 6

### 2-(2,2-Diphenylvinyl)-3,6-dihydroxy-5-phenyl-1,4-benzoquinone

The expected product is obtained in accordance with the procedure described in Example 1 with the replacement of cinnamaldehyde with 3,3-diphenylacrylaldehyde in Step D.

Melting point: 212-213° C.

### EXAMPLE 7

### 2,5-Dihydroxy-3-[(E)-2-(2-naphthyl)-ethenyl]-6-phenyl-1,4-benzoquinone

The expected product is obtained in accordance with the procedure described in Example 1 with the replacement of cinnamaldehyde with (2E)-3-(2-naphthyl)-2-propenal in Step D.

Melting point: 263-264° C.

### EXAMPLE 8

### 2,5-Dihydroxy-3-phenyl-6-(2-phenylcyclopropyl)-1,4-benzoquinone

The expected product is obtained in accordance with the procedure described in Example 1 with the replacement of cinnamaldehyde with 2-phenylcyclopropane carbaldehyde in Step D.

### EXAMPLE 9

### 2,5-Dihydroxy-3-phenyl-6-[(Z)-2-phenylethenyl]-1,4-benzoquinone

The expected product is obtained in accordance with the procedure described in Example 1 with the replacement of cinnamaldehyde with (2Z)-3-phenyl-2-propenal in Step D.

### EXAMPLE 10

### 2,5-Dihydroxy-3-[(E)-1-methyl-2-phenylethenyl]-6-phenyl-1,4-benzoquinone

The expected product is obtained in accordance with the procedure described in Example 1 with the replacement of cinnamaldehyde with (2E)-2-methyl-3-phenyl-2-propenal in Step D.

Melting point: 205-206° C.

### EXAMPLE 11

### 2,5-Dihydroxy-3-phenyl-6-[(1E)-2-phenyl-1-propenyl]-1,4-benzoquinone

The expected product is obtained in accordance with the procedure described in Example 1 with the replacement of cinnamaldehyde with (2E)-3-phenyl-2-butenal in Step D.

Melting point: 227-228° C.

### EXAMPLE 12

### 2,5-Diacetoxy-3-phenyl-6-[(E)-2-phenylethenyl]-1,4-benzoquinone

100 mmol of acetic anhydride are added dropwise, at 0° C., to 10 mmol of the compound of Example 1 in solution in pyridine, and then the reaction mixture is brought to ambient temperature. After stirring for one hour, the reaction mixture is poured onto ice and then extracted with dichloromethane. The organic phase is washed, dried, filtered and then evaporated, and the residue obtained is purified by chromatography on silica (dichloromethane/ethanol 98/2) to yield the expected product.

### EXAMPLE 13

### 2-(4-Chlorophenyl)-3,6-dihydroxy-5-[(E)-2-phenylethenyl]-1,4-benzoquinone

### Step A: 1-(4-Chlorophenyl)-3-hydroxyacetone

There are added slowly to 10 mmol of (4-chlorophenyl)acetyl chloride 25 mmol of tris(trimethylsilyloxy)ethylene and then a few drops of TiCl₄. After stirring for 3 hours at ambient temperature, 14.5 ml of a 3/7 mixture of 0.6M hydrochloric acid and dioxane are added. The reaction mixture is then heated at 90° C. for 10 min, and subsequently brought to ambient temperature. After extraction, the combined organic phases are washed, dried and then evaporated, and the residue obtained is purified by chromatography on silica (eluant:ether/petroleum ether 6/4) and then recrystallised to yield the expected product.

### Step B: 2-(4-Chlorophenyl)-3,6-dihydroxy-5-[(E)-2-phenylethenyl]-1,4-benzoquinone

The expected product is obtained in accordance with the procedure described in Steps B to D of Example 1, starting from the compound obtained in the above Step.

Melting point: 258-259° C.

### EXAMPLE 14

### 2,5-Dihydroxy-3-(4-phenoxyphenyl)-6-[(E)-2-phenylethenyl]-1,4-benzoquinone

The expected product is obtained in accordance with the procedure described in Example 13 with the replacement of (4-chlorophenyl)acetyl chloride with (4-phenoxyphenyl)acetyl chloride in Step A.

### EXAMPLE 15

### 2,5-Dihydroxy-3-[(E)-2-phenylethenyl]-6-(2-pyridyl)-1,4-benzoquinone

The expected product is obtained in accordance with the procedure described in Example 13 with the replacement of (4-chlorophenyl)acetyl chloride with 2-pyridylacetyl chloride in Step A.

### EXAMPLE 16

### 2-(4-Chlorophenyl)-3,6-dihydroxy-5-(2-naphthyl)-1,4-benzoquinone

### Step A: 4-(4-Chlorophenyl)-3-hydroxy-pyran-2,5-dione

The procedure is as in Steps B and C of Example 1, using as starting material the compound obtained in Step A of Example 13.

### Step B: 4-(4-Chlorophenyl)-3-hydroxy-6-(2-naphthylmethylene)-pyran-2,5-dione

A solution of 0.922 mmol of the compound obtained in Step A and 0.922 mmol of 2-naphthaldehyde in 2.45 ml of glacial acetic acid is heated at 60° C. until dissolution occurs. A few drops of concentrated hydrochloric acid are added, the temperature is brought to 90° C. and the reaction mixture is stirred for 6 hours. After cooling to ambient temperature, the flask is plunged into an ice bath and 10 ml of a mixture of diethyl ether and hexane (1/1) are added. The title product is obtained in the form of a yellow powder after filtration.

Melting point: 274-275° C.

### Step C: 2-(4-Chlorophenyl)-3,6-dihydroxy-5-(2-naphthyl)-1,4-benzoquinone

6 ml of a 30% by weight sodium methanolate solution in methanol are added at ambient temperature to a suspension of 0.66 mmol of the compound obtained in Step B in a minimum of anhydrous methanol. After stirring for 1 hour, the reaction mixture is slowly poured into 40 ml of a 1M hydrochloric acid solution cooled beforehand to 0° C. The precipitate obtained is filtered off, washed with water and dried overnight using a dessicator. The title product is obtained in the form of a chestnut-brown powder after recrystallisation from a tetrahydrofuran/hexane mixture.

Melting point: 302-303° C.

### EXAMPLE 17

### 2,5-Dihydroxy-3,6-di-(2-naphthyl)-1,4-benzoquinone

### Step A: (Naphth-2-yl)acetyl Chloride

A mixture of 30 mmol of naphth-2-ylacetic acid and 5 ml of thionyl chloride is heated at reflux under an argon atmosphere for 12 hours. After cooling the reaction mixture and evaporation, the residue obtained is taken up in anhydrous methylene chloride until the excess of thionyl chloride has been eliminated, to yield the title product in the form of a yellow oil.

### Step B: 1-Hydroxy-3-(2-naphthyl)acetone

At ambient temperature, under a stream of argon, 71.45 mmol of tris(trimethylsilyloxy)ethylene are slowly added to 28.58 mmol of (naphth-2-yl)acetyl chloride (obtained in Step A). After stirring for 5 hours at 90° C., the mixture is cooled and then a mixture of 12 ml of 0.6M hydrochloric acid and 30 ml of dioxane is slowly added. The reaction mixture is heated at 90° C. for 10 minutes and then cooled and extracted several times with diethyl ether. The organic phases are combined and washed in succession with a saturated aqueous solution of sodium hydrogen carbonate and a saturated aqueous solution of sodium chloride. The organic phase is dried over sodium sulphate, filtered and concentrated using a rotary evaporator. The residue obtained is chromatographed on silica gel using as eluant an ethyl acetate/hexane mixture in the proportions 6/4.

The title product is obtained after recrystallisation from hexane.

Melting point: 109-110° C.

### Step C: 3-Hydroxy-4-(2-naphthyl)-pyran-2,5-dione

The procedure is as in Steps B and C of Example 1, starting from the compound obtained in Step B.

The title compound is obtained in the form of a yellow powder after recrystallisation from a CH₂Cl₂/hexane mixture.

Melting point: 196-197° C.

### Step D: 2,5-Dihydroxy-3,6-di(2-naphthyl)-1,4-benzoquinone

The procedure is as in Steps B and C of Example 16, starting from the compound obtained in Step C.

The title compound is obtained in the form of a deep-pink powder after recrystallisation from a tetrahydrofuran/hexane mixture.

Melting point: 328-329° C.

### EXAMPLE 18

### 2-(4-Fluorophenyl)-3,6-dihydroxy-5-(2-naphthyl)-1,4-benzoquinone

The procedure is as in Example 13 starting from (4-fluorophenyl)acetyl chloride and naphthaldehyde.

Chestnut-brown powder.

Melting point: 300-301° C.

### EXAMPLE 19

### 2-(4-Bromophenyl)-3,6-dihydroxy-5-(2-naphthyl)-1,4-benzoquinone

The procedure is as in Example 13 starting from (4-bromophenyl)acetyl chloride and naphthaldehyde.

Chestnut-brown powder.

Melting point: 312-313° C.

### EXAMPLE 20

### 2-(2-Chlorophenyl)-3,6-dihydroxy-5-(2-naphthyl)-1,4-benzoquinone

The procedure is as in Example 13 starting from (2-chlorophenyl)acetyl chloride and naphthaldehyde.

Chestnut-brown powder.

Melting point: 242-243° C.

### EXAMPLE 21

### 2-(6-Bromo-2-naphthyl)-3,6-dihydroxy-5-phenyl-1,4-benzoquinone

The procedure is as in Example 1 starting from allylbenzene and (6-bromo)-2-naphthaldehyde.

### EXAMPLE 22

### 2,5-Dihydroxy-3-(2-naphthyl)-6-[3-(trifluoromethyl)phenyl]-1,4-benzoquinone

The procedure is as in Example 13 starting from (3-trifluoromethylphenyl)acetyl chloride and naphthaldehyde.

### EXAMPLE 23

### 2,5-Dihydroxy-3-(2-naphthyl)-6-(4-nitrophenyl)-1,4-benzoquinone

The procedure is as in Example 13 starting from (4-nitrophenyl)acetyl chloride and naphthaldehyde.

### EXAMPLE 24

### 2-(4-Bromophenyl)-3,6-dihydroxy-5-(6-methoxy-2-naphthyl)-benzo-1,4-quinone

The procedure is as in Example 13 starting from (4-bromophenyl)acetyl chloride and (6-methoxy)-2-naphthaldehyde.

### EXAMPLE 25

### 2,5-Diacetoxy-3-(4-Bromophenyl)-6-(2-naphthyl)-1,4-benzoquinone

The procedure is as in Example 12 starting from the compound obtained in Example 19.

### EXAMPLE 26

### 2-(3-Chlorophenyl)-3,6-dihydroxy-5-(2-naphthyl)-1,4-benzoquinone

The procedure is as in Example 13 starting from (3-chlorophenyl)acetyl chloride and naphthaldehyde.

Melting point: 258-259° C.

### EXAMPLE 27

### 2-[(E)-2-(4-Fluorophenyl)ethenyl]-3,6-dihydroxy-5-phenyl-1,4-benzoquinone

The procedure is as in Example 1 with the replacement of cinnamaldehyde with 4-fluoro-cinnamaldehyde in Step D.

Melting point: 250-251° C.

### EXAMPLE 28

### 2-(4-Bromophenyl)-3,6-dihydroxy-5-[(E)-2-phenylethenyl]-1,4-benzoquinone

The procedure is as in Example 13 using as starting material (4-bromophenyl)acetyl chloride.

Melting point: 262-263° C.

### EXAMPLE 29

### 2-(2-Chlorophenyl)-3,6-dihydroxy-5-[(E)-2-phenylethenyl]-1,4-benzoquinone

The procedure is as in Example 13 using as starting material (2-chlorophenyl)acetyl chloride.

Melting point: 266-268° C.

### EXAMPLE 30

### 2-(3-Chlorophenyl)-3,6-dihydroxy-5-[(E)-2-phenylethenyl]-1,4-benzoquinone

The procedure is as in Example 13 using as starting material (3-chlorophenyl)acetyl chloride.

Melting point: 210-211° C.

### EXAMPLE 31

### 2,5-Dihydroxy-3-[(E)-1-methyl-2-phenylethenyl]-6-(2-naphthyl)-1,4-benzoquinone

The procedure is as in Steps D and E of Example 1 starting from the compound obtained in Step C of Example 17 and with the replacement of cinnamaldehyde with (2E)-2-methyl-3-phenyl-2-propenal in Step D.

Melting point: 217-218° C.

### EXAMPLE 32

### 2,5-Dihydroxy-3-(2-naphthyl)-6-[(E)-2-phenylethenyl]-1,4-benzoquinone

The procedure is as in Steps D and E of Example 1 starting from the compound obtained in Step C of Example 17.

Melting point: 247-248° C.

### EXAMPLE 33

### 2-(4-Chlorophenyl)-5-(2,2-diphenylvinyl)-3,6-dihydroxy-1,4-benzoquinone

The procedure is as in Example 13 starting from (4-chlorophenyl)acetyl chloride and 3,3-diphenylacrylaldehyde.

Melting point: 253-254° C.

### EXAMPLE 34

### 2-(4-Fluorophenyl)-3,6-dihydroxy-5-[(E)-2-phenylethenyl-1,4-benzoquinone

The procedure is as in Example 13 using as starting material (4-fluorophenyl)acetyl chloride.

Melting point: 259-260° C.

### EXAMPLE 35

### 2,5-Dihydroxy-3-(3-methylphenyl)-6-[(E)-2-phenylethenyl]-1,4-benzoquinone

The procedure is as in Example 13 using as starting material (3-methylphenyl)acetyl chloride.

### EXAMPLE 36

### 2-(4-Ethylphenyl)-5-[(E)-2-(4-fluorophenyl)ethenyl]-3,6-dihydroxy-1,4-benzoquinone

The procedure is as in Example 13 starting from (4-ethylphenyl)acetyl chloride and 4-fluoro-cinnamaldehyde.

### EXAMPLE 37

### 2,5-Dihydroxy-3-[(E)-2-(4-methylphenyl)ethenyl]-6-(4-nitrophenyl)-1,4-benzoquinone

The procedure is as in Example 13 starting from (4-nitrophenyl)acetyl chloride and 4-methyl-cinnamaldehyde.

### EXAMPLE 38

### 2-(4-Fluorophenyl)-3,6-dihydroxy-5-[(E)-2-(3-propoxyphenyl)-ethenyl]-1,4-benzoquinone

The procedure is as in Example 13 starting from (4-fluorophenyl)acetyl chloride and 3-propoxy-cinnamaldehyde.

### EXAMPLE 39

### 2,5-Dihydroxy-3-phenyl-6-{(E)-2-[4-(trifluoromethyl)phenyl]-ethenyl}-1,4-benzoquinone

The procedure is as in Example 1 with the replacement of cinnamaldehyde with 4-trifluoromethyl-cinnamaldehyde in Step D.

### EXAMPLE 40

### 2-[4-Bromophenyl]-3,6-dihydroxy-5-phenyl-1,4-benzoquinone

The procedure is as in Example 1 with the replacement of cinnamaldehyde with 4-bromobenzaldehyde in Step D.

Melting point: 250-251]C.

### Pharmacological Study of the Compounds of the Invention

### EXAMPLE A

### Activation of Insulin Receptors (IR) and Protein Kinase B (PKB)

The pharmacological effect of the compounds of the invention on cellular signaling is evaluated in vitro using hamster ovary cells transfected with human insulin receptors (CHO-HIR). The techniques employed are from TAVARE and DENTON (1988, BIOCHEM. J., 250, 509-519) and TAVARE et al. (1988, Biochem. J., 253, 783-788) modified by ISSAD et al. (1991, Biochem. J., 275, 15-21) and COMBETTES-SOUVERAIN et al. (1997, Diabetologia, 40, 533-540).

The products are studied at 10⁻⁵M under incubation for two hours at 37° C. with CHO-HIR in culture. A negative control (solvent) and a positive control (insulin 50 nM, 5 min incubation) are used in parallel in the same test. At the end of the incubation periods, the enzymatic reactions are immediately stopped by a mixture of protease inhibitors (aprotinin, pepstatin, antipaine, leuleptin, AEBSF) and the samples are plunged into ice at 4° C.

The phosphorylation of the insulin receptors (IR) and the phosphorylation of protein kinase B are evaluated by immunoblotting as follows.

The IR are extracted over wheatgerm lectin; the samples are then subjected to electrophoresis on 7.5% polyacrylamide gel and subjected to an electric transfer onto PDVF membrane in a semi-dry system. After blocking, the membranes are incubated with an antiphosphotyrosine antibody (p-tyr(PY99), ref SC7020, Santa Cruz) and the chemiluminescence associated with a conjugated antibody is detetected by a LAS1000 camera (Fujifilm). After dehydridisation and then rehydridisation with an anit-insulin receptor β subunit antibody (βsubunit, ref 06492, Upstate Biotechnology), the total IR quantity deposited on blot is evaluated by chemiluminescence and the phosphorylation rate of the receptors is related to the total receptor quantity deposited.

Similarly, the state of phosphorylation of protein kinase B following electrophoresis of the samples on 10% polyacrylamide gel is determined by chemiluminescence after immunoblotting with anti-phospho protein kinase B antibodies (phospho Akt(Ser473) antibody, ref 9271, Cell Signaling) and then related to the total protein kinase B quantity (AKT antibody, ref 9272, Cell signaling).

The phosphorylation induced by the compounds of the invention is expressed as a percentage in relation to 50 nM insulin, fixed at 100% activation.

The compounds of the invention activate either mostly insulin receptors or mostly protein kinase B, or activate both simultaneously, demonstrating their potential activity as insulinomimetic compounds.

By way of example, the compound of Example 27, at 10⁻⁵M, has an activation percentage of 87.7% (n=1) for insulin receptors and of 48.9% (n=1) for proetin kinase B.

The compound of Example 10, at 10⁻⁵M, has an activation percentage of 91% (n=3) for protein kinase B. The compound of Example 30, at 10⁻⁵M, has an activation percentage of 134.4% (n=2) for insulin receptors.

### EXAMPLE B

### Chronic In Vivo Study

11-week-old female ob/ob C57BL/6 mice randomly selected in terms of basal glycaemia from animals that have been fed are treated for 9 days with the test compound at 10 or 20 mg/kg po and compared with a control group receiving 1% HEC. Biological balance (glycaemia, insulinaemia, triglyceridaemia) is reached on the 10th day with the animals that have been fed, the final treatment having taken place the day before. The results are expressed as percentage variations in relation to D0 (glycaemia, body weight) compared with the control group.

The results obtained demonstrate that the body weight is appreciably reduced after treatment with the compounds of the invention. In particular, the compound of Example 16 exhibits a decrease of −26.9% whereas the result obtained for the control group is +4.9%. It is to be noted that the effect observed is dose-dependent.

The glycaemia and the insulinaemia are likewise appreciably reduced on treatment with the compounds of the invention. For example, for the compound of Example 16 a result of −60.7% was recorded compared with +4.5% for the controls for glycaemia, and −84.2% compared with the controls for insulinaemia.

The results obtained also show a decrease in triglycerides on treatment with the compounds of the invention: by way of example, the compound of Example 1 shows a reduction of −37.8% compared with the control.

Those results attest to the excellent in vivo activity of the compounds according to the invention as insulinomimetic agents for use in that capacity in the treatment of diabetes.

### EXAMPLE C

### Pharmaceutical Composition

Formulation for the preparation of 1000 tablets each
comprising a dose of 10 mg

| | |
|---|---|
| compound of Example 1 | 10 g |
| hydroxypropyl cellulose | 2 g |
| wheat starch | 10 g |
| lactose | 100 g |
| magnesium stearate | 3 g |
| talc | 3 g |

1-19. (canceled)

20. A compound selected from those of formula (1): 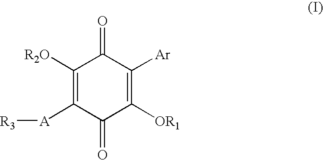
wherein:
R₁ and R₂, which may be identical or different, each represents hydrogen, linear or branched (C₁-C₆)acyl or linear or branched (C₁-C₆)alkyl,
Ar represents aryl or heteroaryl,
A represents a group selected from: 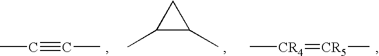
wherein,
R₄ substitutes the carbon atom bonded to the benzoquinone ring and represents hydrogen or linear or branched (C₁-C₆)alkyl, and
R₅ substitutes the carbon atom bonded to the radical R₃ and represents hydrogen or a group selected from linear or branched (C₁-C₆)alkyl, aryl and heteroaryl,
R₃ represents aryl, heteroaryl, dicyclopropylmethyl or benzhydryl,
or A-R₃ represents an optionally substituted naphthyl group, and in that case Ar represents aryl,
and its stereoisomers, where they exist, and addition salts thereof with a pharmaceutically acceptable acid or base,
provided that the compound of formula (I) is not 2,5-dihydroxy-3-(4-methoxyphenyl)-6-(2-phenylethenyl)-1,4-benzoquinone, 2,5-dihydroxy-3-(4-methoxyphenyl)-6-(2-naphthyl)-1,4-benzoquinone or 2,5-dihydroxy-3-(2-naphthyl)-6-phenyl-1,4-benzoquinone,
it being understood that:
aryl means phenyl, biphenylyl, naphthyl or tetrahydronaphthyl, each of those groups optionally being substituted by one or more identical or different atoms or groups selected from halogen, linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl, (C₁-C₂)alkylenedioxy and phenyloxy,
an optionally substituted naphthyl group means a naphthyl group that is unsubstituted or substituted by one or more identical or different atoms or groups selected from halogen, linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl, (C₁-C₂)alkylenedioxy and phenyloxy,
heteroaryl means an aromatic mono- or bi-cyclic group having from 5 to 12 ring members and containing one, two or three hetero atoms selected from oxygen, nitrogen and sulphur, wherein the heteroaryl may optionally be substituted by one or more identical or different atoms or groups selected from halogen, linear or branched (C₁-C₆)alkyl, hydroxy, linear or branched (C₁-C₆)alkoxy, linear or branched (C₁-C₆)-polyhaloalkyl, amino (optionally substituted by one or more linear or branched (C₁-C₆)alkyl groups), nitro, linear or branched (C₁-C₆)acyl, (C₁-C₂)alkylenedioxy and phenyloxy.

21. A compound of claim 20, wherein:
R₃ represents aryl, heteroaryl, dicyclopropylmethyl or benzhydryl,
A represents a group selected from: 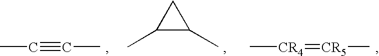
wherein:
R₄ substitutes the carbon atom bonded to the benzoquinone ring and represents hydrogen or linear or branched (C₁-C₆)alkyl, and
R₅ substitutes the carbon atom bonded to the radical R₃ and represent hydrogen or a group selected from linear or branched (C₁-C₆)alkyl, aryl and heteroaryl,
and its stereoisomers, where they exist, and addition salts thereof with a pharmaceutically acceptable acid or base.

22. A compound of claim 20, wherein:
Ar represents aryl, and
A-R₃ represents an optionally substituted naphthyl group,
and its stereoisomers, where they exist, and also addition salts thereof with a pharmaceutically acceptable acid or base.

23. A compound of claim 20 wherein R₁ and R₂ each represents hydrogen, its stereoisomers and addition salts thereof with a pharmaceutically acceptable acid or base.

24. A compound of claim 20 wherein Ar represents aryl, its stereoisomers and addition salts thereof with a pharmaceutically acceptable acid or base.

25. A compound of claim 20 wherein A-R₃ represents arylethenyl, its stereoisomers and addition salts thereof with a pharmaceutically acceptable acid or base.

26. A compound of claim 20 which is 2-(4-chlorophenyl)-3,6-dihydroxy-5-(2-naphthyl)-1,4-benzoquinone, and its addition salts with a pharmaceutically acceptable acid or base.

27. A compound of claim 20 which is 2-(4-bromophenyl)-3,6-dihydroxy-5-(2-naphthyl)-1,4-benzoquinone, and its addition salts with a pharmaceutically acceptable acid or base.

28. A compound of claim 20 which is 2-[(E)-2-(4-fluorophenyl)ethenyl]-3,6-dihydroxy-5-phenyl-1,4-benzoquinone, and its addition salts with a pharmaceutically acceptable acid or base.

29. A compound of claim 20 which is 2-(4-chlorophenyl)-3,6-dihydroxy-5-[(E)-2-phenylethenyl]-1,4-benzoquinone, and its addition salts with a pharmaceutically acceptable acid or base.

30. A compound of claim 20 which is 2,5-dihydroxy-3-phenyl-6-[(E)-2-phenylethenyl]-1,4-benzoquinone, and its addition salts with a pharmaceutically acceptable acid or base.

31. A compound of claim 20 which is 2-(4-bromophenyl)-3,6-dihydroxy-5-[(E)-2-phenylethenyl]-1,4-benzoquinone, and also its addition salts with a pharmaceutically acceptable acid or base.

32. A compound of claim 20 which is 2,5-dihydroxy-3-[(E)-1-methyl-2-phenylethenyl]-6-phenyl-1,4-benzoquinone, and its addition salts with a pharmaceutically acceptable acid or base.

33. A compound of claim 20 which is 2-(3-chlorophenyl)-3,6-dihydroxy-5-[(E)-2-phenylethenyl]-1,4-benzoquinone, and its addition salts with a pharmaceutically acceptable acid or base.

34. A compound of claim 20 which is 2-(4-bromophenyl)-3,6-dihydroxy-5-phenyl-1,4-benzoquinone, and also its addition salts with a pharmaceutically acceptable base.

35. A pharmaceutical composition comprising as active ingredient a compound of claim 20 in combination with one or more pharmaceutically acceptable, inert, non-toxic carriers.

36. A method of treating a living animal body, including a human, afflicted with a condition associated with a deregulation of glycaemia, comprising the step of administering to the living animal body, including a human an amount of a compound of claim 20 which is effective for alleviation of the condition.

37. A method of treating a living animal body, including a human, afflicted with diabetes, comprising the step of administering to the living animal body, including a human, an amount of a compound of claim 20 which is effective for alleviation of diabetes.
